(19) United States
(12) Patent Application Publication
(10) Pub. No.: US 2025/0000690 A1
(43) Pub. Date: Jan. 2, 2025

(54) PERSONAL COOLING APPARATUSES WITH NECK COLLARS, AND RELATED METHODS OF USE

(71) Applicant: Claude Garneau, Edmonton (CA)

(72) Inventor: Claude Garneau, Edmonton (CA)

(21) Appl. No.: 18/757,128

(22) Filed: Jun. 27, 2024

(30) Foreign Application Priority Data
Jun. 27, 2023 (CA) 3205215

(51) Int. Cl. A61F 7/00; A45F 3/04; A45F 3/16

(52) U.S. Cl. CPC A61F 7/00; A45F 3/04; A45F 3/16; A45F 2003/166; A61F 2007/0011; A61F 2007/0056

(57) ABSTRACT

A personal cooling apparatus has an air mover; a drinking fluid reservoir; an air conduit connected to an outlet of the air mover and configured for heat exchange with the drinking fluid reservoir; and a neck collar tube connected to the air conduit and defining a series of outlet openings spaced about a periphery of the neck collar tube.

### TECHNICAL FIELD

This document relates to personal cooling apparatuses, and related methods of use.

### BACKGROUND

The following paragraphs are not an admission that anything discussed in them is prior art or part of the knowledge of persons skilled in the art.

Backpack-mounted personal cooling apparatuses exist with shoulder straps, pumps, and air outlets in close proximity with a user's face, to expel cooling air onto the user's face during use.

### SUMMARY

A personal cooling apparatus is disclosed comprising: an air mover; a drinking fluid reservoir; an air conduit connected to an outlet of the air mover and configured for heat exchange with the drinking fluid reservoir; and a neck collar tube connected to the air conduit and defining a series of outlet openings spaced about a periphery of the neck collar tube.

A method is disclosed comprising operating an air mover, of a personal cooling apparatus worn by a user, to: move air from an external environment into heat exchange with a drinking fluid reservoir, of the personal cooling apparatus, to cool the air; and expel the air upward around a face of the user via a series of outlet openings spaced about a periphery of a neck collar tube, of the personal cooling apparatus.

In various embodiments, there may be included any one or more of the following features: The neck collar tube defines a ring. The neck collar tube comprises latex. The series of outlet openings are spaced about a top periphery of the neck collar tube. The series of outlet openings are spaced about an inner periphery of the neck collar tube. The personal cooling apparatus is configured to expel a vertical column of air, from the series of outlet openings, about a user's head in use. The air conduit forms an envelope that encloses the drinking fluid reservoir, with inner surfaces of the envelope and outer surfaces of the drinking fluid reservoir defining a peripheral air passage plenum around the drinking fluid reservoir. The drinking fluid reservoir comprises one or more bottles. The envelope is shaped to form a conformal enclosure around the one or more bottles. The one or more bottles comprise two or more cylindrical bottles. The envelope forms a clamshell that encloses the two or more cylindrical bottles when in a closed position. The air conduit is configured for non-mixing heat exchange with the drinking fluid reservoir. The air conduit is at least partially inset within an indented conduit slot defined by an exterior wall of the drinking fluid reservoir. The air conduit comprises an aluminum tube. The air conduit comprises a rigid standpipe that connects via a manifold to the neck collar tube. The manifold has one or more outlets, at least one of which feeds the neck collar tube. The manifold comprises a fitting with first and second outlets feeding first and second ends of the neck collar tube. At least one outlet of the one or more outlets is connected to feed a body cooling tube. The manifold comprises a valve. A drinking fluid tube is connected to the drinking fluid reservoir. One or more drinking fluid tubes are connected to the drinking fluid reservoir. The drinking fluid reservoir comprises one or more water filters. The air mover or air conduit comprise one or more air filters. A housing mounts the air mover, drinking fluid reservoir, and air conduit. The housing comprises a backpack with shoulder straps. A controller is provided for the air mover. The drinking fluid reservoir contains ice. Operating the air mover of the personal cooling apparatus to pump air through the personal cooling apparatus to cool a user.

The foregoing summary is not intended to summarize each potential embodiment or every aspect of the subject matter of the present disclosure. These and other aspects of the device and method are set out in the claims.

### BRIEF DESCRIPTION OF THE FIGURES

Embodiments will now be described with reference to the figures, in which like reference characters denote like elements, by way of example, and in which:

FIG. 1 is cross-section side view of a personal cooling apparatus within a backpack housing.

FIG. 2 is perspective view of the personal cooling apparatus of FIG. 1 with the backpack housing being shown in dashed lines.

FIG. 3 is a side elevation view of the personal cooling apparatus of FIG. 1 worn by a user.

FIG. 4 is a rear perspective view of the personal cooling apparatus of FIG. 1 being worn by a user.

FIG. 5 is a front perspective view of the personal cooling device of FIG. 1 being worn by a user.

FIG. 6 is a perspective view of a manifold of the personal cooling apparatus of FIG. 1.

FIG. 7 is a perspective view of a cap used to seal a third outlet of the manifold of FIG. 6 when the third outlet is not in use.

FIG. 8 is a perspective view of an air inlet in a shirt which connects to the third outlet of the manifold of FIG. 6 to provide air flow to a user's body.

FIG. 9 is a perspective view of an air tube which connects the third outlet of the manifold of FIG. 6 to provide manual control of the cooling air by a user.

FIG. 10 is a bottom plan view of the manifold of FIG. 6 with a valve for the third outlet closed.

FIG. 11 is a bottom plan view of the manifold of FIG. 6 with the valve for the third outlet open.

FIG. 12 is a schematic view of the air mover, power source, and controls.

FIG. 13 is a top plan view of a neck collar tube of the personal cooling apparatus of FIG. 1.

FIG. 14 is a rear perspective view of a personal cooling device, within a backpack, with a pouch for a drinking reservoir and a pouch for a battery open, and the drinking reservoir and battery removed from the respective pouches.

FIG. 15 is an exploded view of the manifold of FIG. 6 and an air inlet in a shirt of a user.

FIG. 16 is a front perspective view of the personal cooling device of FIG. 4, without straps, illustrating optional texturing on the front side (user's back-contacting side) of the backpack.

FIG. 17 is a rear elevation view of a personal cooling apparatus within a backpack housing, with a rear half of the housing removed to display the inner components therein.

FIG. 18 is the same rear elevation view of FIG. 17 with the rear half of the housing installed to enclose the inner components.

FIG. 19. is a side elevation view of the personal cooling apparatus of FIG. 18.

FIG. 20 is a rear elevation view of the envelope air conduit module of the personal cooling apparatus of FIG. 17.

FIG. 21 is a view taken along the 21-21 cross-section lines of FIG. 20, illustrating the envelope in a closed position in solid lines, overlaid with the envelope and a pair of ice bottles in dashed lines in an exploded configuration to show an open position of the envelope configured to receive or release the ice bottles.

### DETAILED DESCRIPTION

Immaterial modifications may be made to the embodiments described here without departing from what is covered by the claims.

Climate change refers to the theory of long-term alteration of temperature and weather patterns on Earth, largely attributed to human activities such as the burning of fossil fuels and deforestation. One significant consequence of climate change is the increasing trend of hotter weather worldwide. Rising concentrations of greenhouse gases, such as carbon dioxide and methane, trap more heat in the atmosphere, leading to a gradual warming of the planet. As a result, average global temperatures have been steadily rising over the past century. This warming effect manifests in various ways, including heatwaves, prolonged periods of high temperatures, and a general increase in extreme weather events. The impacts of hotter weather are far-reaching and can include detrimental effects on ecosystems, agriculture, human health, and the overall functioning of the planet. It may be crucial to address climate change and implement sustainable solutions to mitigate its effects, as a hotter climate poses significant challenges for the well-being of both humans and the environment.

Being outside in extreme hot weather poses significant challenges and potential risks to human health and well-being. When exposed to high temperatures, the body's natural cooling mechanisms, such as sweating, may struggle to keep up with the heat. This may lead to heat-related illnesses, ranging from heat exhaustion to heatstroke, which can be life-threatening. In extreme heat, the body may experience dehydration, electrolyte imbalances, and increased strain on the cardiovascular system. Prolonged exposure to hot weather may also exacerbate existing medical conditions, such as respiratory and cardiovascular diseases. Additionally, the combination of high temperatures and high humidity levels can make it difficult for the body to dissipate heat effectively, further increasing the risk of heat-related ailments. It may be crucial to take precautions when being outside in extreme hot weather, such as staying hydrated, wearing lightweight and breathable clothing, seeking shade or air-conditioned environments, and limiting outdoor activities during peak heat hours.

Prolonged outdoor exposure to extreme heat can significantly increase the risk of various health issues. In some cases, a user may be more likely to experience heat stroke, a severe heat-related illness that can have life-threatening consequences. Heat stroke may occur when the body's core temperature rises above 40 degrees Celsius (104 degrees fahrenheit) due to prolonged exposure to high temperatures and inadequate thermoregulation. Heat stroke is characterized by symptoms such as high body temperature, rapid heartbeat, dizziness, confusion, nausea, and even loss of consciousness. Heat stroke is a severe type of heat stress and a medical emergency that requires immediate attention. If left untreated, heat stroke can lead to organ damage, including brain damage, and in severe cases, it can be fatal. Individuals working in hot climates should be vigilant and aware of the signs of heat stroke, both in themselves and their colleagues. Prevention of heat stroke is key, and measures such as taking frequent breaks in shaded or cool areas, wearing appropriate protective clothing, staying hydrated, and monitoring the body's response to heat can help reduce the risk. It is essential to prioritize safety, seek medical assistance if symptoms of heat stroke occur, and take necessary steps to mitigate the dangers of heat stroke in extreme heat conditions.

In extreme hot weather conditions, individuals may experience a phenomenon that can be referred to as cranial overheating, characterized by the elevation of head temperature beyond normal limits. This condition can result in a range of distressing symptoms and potentially lead to various medical complications. Initial signs may include intense cephalalgia, commonly referred to as a severe headache, often accompanied by throbbing sensations. As cranial temperature continues to rise, individuals may experience dizziness, vertigo, and disorientation, impairing their cognitive functions and spatial awareness. Profuse sweating, flushed skin, and an increased heart rate may also manifest as the body attempts to regulate its internal temperature. In severe cases, hyperthermia can develop, where the core body temperature rises dangerously, potentially leading to heat stroke, seizures, or loss of consciousness.

Working outside in a hot climate presents additional challenges that can impact both productivity and the health of individuals. Jobs requiring physical labor in extreme heat can strain the body's thermoregulatory system, leading to increased fatigue, decreased concentration, and diminished overall performance and, over long periods of exposure, life expectancy. In such situations, it may be even more important to implement proper heat management strategies.

In extreme heat, water bottles and backpack bladder systems may be used to combat the potential risks of dehydration and heat-related illnesses, such as heat stroke. Water bottles provide a convenient and portable means of carrying water during outdoor activities. Many are made of materials that are suitable for extreme temperatures, such as insulated stainless steel or BPA-free plastic, to keep the water cool for longer periods. Additionally, backpack bladder systems, also known as hydration reservoirs, offer a hands-free and efficient way to stay hydrated during extended periods of outdoor work or physical activity. Such systems may include a bladder that can hold a relatively large volume of water, typically ranging from 1.5 to 3 liters, and a drinking tube with a bite valve for easy access to water. When using either water bottles or backpack bladder systems, it may be essential to an individual to drink water regularly and stay hydrated by consuming fluids at frequent intervals, even before feeling thirsty. In extremely hot conditions, it may be necessary to increase water intake to compensate for increased sweat loss and maintain proper hydration levels.

In some cases, a personal cooling vest may be used by a worker in an extremely hot environment, to provide relief and regulate body temperature for individuals working in extremely hot environments. Such vests may incorporate advanced cooling technologies to help maintain thermal comfort and prevent heat-related illnesses. One approach involves the use of phase-change materials (PCM) embedded within the vest. PCM may absorb excess heat from the body, undergoing a phase transition (such as transitioning from a solid to liquid), and absorbing latent heat, creating a cooling effect. These materials may be pre-cooled in a refrigerator or chilled environment, ensuring an extended cooling duration. Other types of personal cooling vests may utilize evaporative cooling, and/or be equipped with specialized fabrics that efficiently wick away sweat and enhance the evaporation process, to cool the body. Evaporative cooling effect may be amplified by incorporating airflow through strategically placed vents or fans within the vest. Personal cooling vests offer adjustable fit options and lightweight designs, allowing case of movement and minimizing discomfort. By leveraging these advanced cooling technologies, personal cooling vests offer a practical solution to enhance the well-being and safety of individuals working in extremely hot conditions.

Personal air conditioning systems may also be used by individuals to combat extreme heat and provide individualized cooling in hot environments. Personal air conditioning systems may be designed to create a localized cool microenvironment around the user. Typically worn as a portable device, a personal air conditioning system may use various cooling mechanisms to deliver cooled air directly to the individual. Some systems may employ miniaturized compressors and refrigeration cycles to cool the air before it is directed towards the user. Other systems may use thermoelectric cooling, where a temperature gradient is generated using the Peltier effect, resulting in the transfer of heat away from the body. Personal air conditioning systems often come with adjustable settings to control the airflow, temperature, and fan speed, allowing users to customize their cooling experience. These compact and portable systems typically include rechargeable batteries for enhanced mobility. Personal air conditioning systems offer an effective and convenient solution to alleviate heat stress and improve comfort for individuals working in hot environments.

In addition to advanced cooling technologies, there are simpler forms of personal cooling that individuals can employ in hot environments. One such method is the use of a portable fan. Fans create airflow that helps evaporate sweat from the skin, promoting natural cooling through evaporation. By directing the airflow towards the body, a fan can provide immediate relief and increase comfort levels. Another option is the use of ice packs or cold compresses. Ice packs or gel-filled packs that have been cooled in a freezer can be placed on pulse points or specific areas of the body, such as the neck, wrists, or forehead. The cold temperature of the ice packs helps to lower the body's surface temperature, providing a temporary cooling sensation. Additionally, individuals can wet a towel or bandana with cool water or wrap ice cubes in a cloth and apply it to the neck or forehead for a similar cooling effect. These simple yet effective methods offer practical and accessible means of personal cooling in hot environments, providing individuals with immediate relief and helping to maintain thermal comfort.

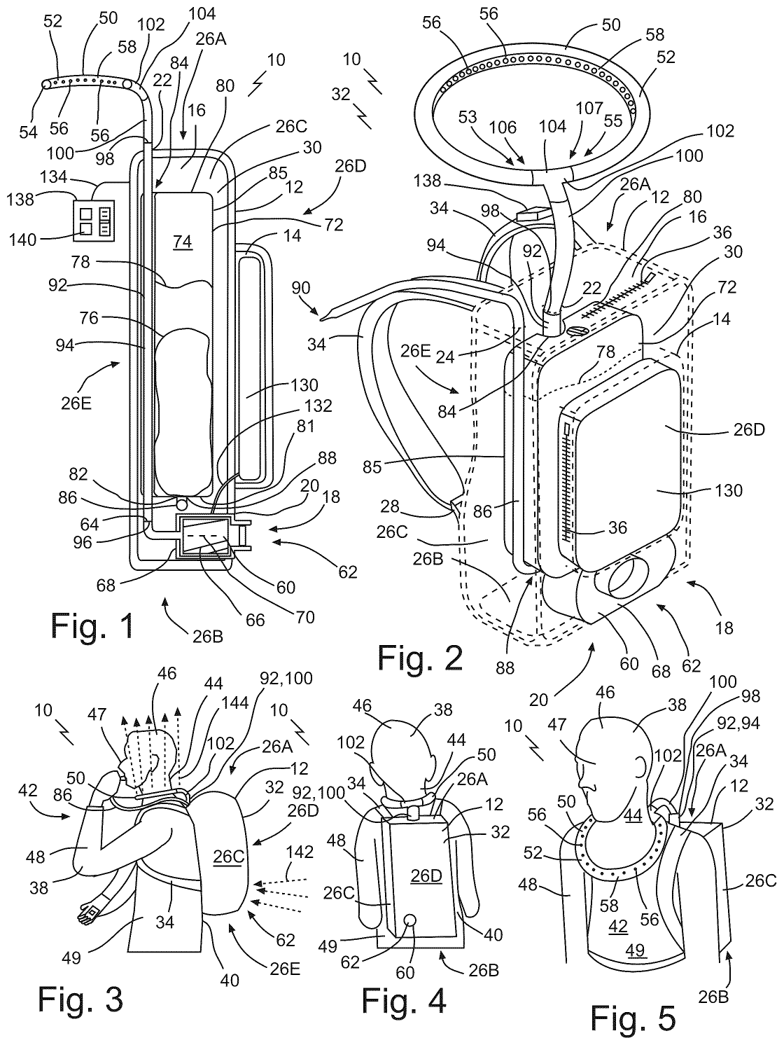

Referring to FIGS. 1-5 and 14, a personal cooling apparatus 10 is disclosed. The personal cooling apparatus 10 may be used to cool a user 38. The personal cooling apparatus 10 comprises an air mover 60, such as incorporating a low-profile or other centrifugal fan. The apparatus 10 may comprise a drinking fluid reservoir 72, which may be used to provide hydration to the user 38. The apparatus 10 comprises an air conduit 92. The air conduit 92 may be connected to an outlet 64 of the air mover 60. The air conduit 92 may be configured for heat exchange with a drinking fluid reservoir 72 to cool the air within the air conduit 92. The apparatus 10 comprises a neck collar tube 50. The neck collar tube 50 may be connected to receive air from the air conduit 92. The neck collar tube 50 may define a series of outlet openings 56 about a top periphery 58 of the neck collar tube 50. The series of outlet openings 56 may in use exhaust cooled air from the neck collar tube 50 towards the user 38. The air mover 60 may draw air from an external environment into the apparatus 10 through an inlet 62 of the air mover 60. The air mover 60 may move air into the air conduit 92. The air mover 60 may continue to move the air into heat exchange with the drinking fluid reservoir 72, through the air conduit 92 and into the neck collar tube 50. The air mover 60 may expel the air upward around a face 47 of the user 38 via the series of outlet openings 56 spaced about the top periphery 58 of the neck collar tube 50.

Referring to FIGS. 1-5, 14 and 16, the personal cooling apparatus 10 may comprise a suitable housing 12, such as a backpack. The housing 12 may mount the air mover 60, drinking fluid reservoir 72 and air conduit 92. The housing 12 may comprise a battery receptacle or pouch 14, such as on a rear face of the housing 12, and which may house a battery 130. The housing 12 may comprise a drinking reservoir receptacle or pouch 16, which may house the drinking fluid reservoir 72. The pouches 14 and 16 may be accessible via recloscable openings 36. The reclosable openings 36 may be formed by a suitable recloseable connectors, for example a zipper, drawstring, or hook and loop connector.

Referring to FIGS. 1-5, 14 and 16, the housing 12 may be a suitable housing to mount the various components of the apparatus 10, such as a backpack 32 with shoulder straps 34. The housing 12 may define a top surface 26A, a bottom surface 26B, side surfaces 26C, a front surface 26D and a rear surface 26E (back-contacting surface). Referring to FIG. 16, the rear surface 26E of the backpack 32 may be textured, for example ridged, to allow for increased air flow between the back 40 of a user 38 and the rear surface 26E of the backpack 32, in some cases defining air channels along the back of the user in use. An increase in airflow between the back 40 of a user 38 and the rear surface 26E of the backpack 32 may promote a higher rate of evaporative cooling. Referring to FIGS. 1-5, 14 and 16, a backpack is a versatile and widely used piece of equipment designed for carrying various items in a convenient and organized manner. In a technical context, a backpack typically refers to a bag that incorporates straps to mount on the back of a user, and features multiple compartments, pockets, and straps for efficient storage and distribution of weight. Backpacks are commonly made from durable materials such as nylon or polyester to withstand heavy loads and rugged use. Backpacks may be designed with ergonomics in mind, incorporating padded shoulder straps and a supportive back panel to distribute weight evenly and enhance comfort during extended wear. Shoulder straps 34 may have suitable parts, such as clasps, buckles, and length adjusters 28, which may allow the user 38 to size the straps 34 appropriately to the user's body 49. Some backpacks also include a hip belt and/or chest strap to further stabilize the load and reduce strain on the shoulders and back. Backpacks may serve as essential tools for a wide range of applications, including outdoor adventures, commuting, work, and education. In other cases, other forms of packs may be used as the housing, such as a shoulder bag, purse, or other.

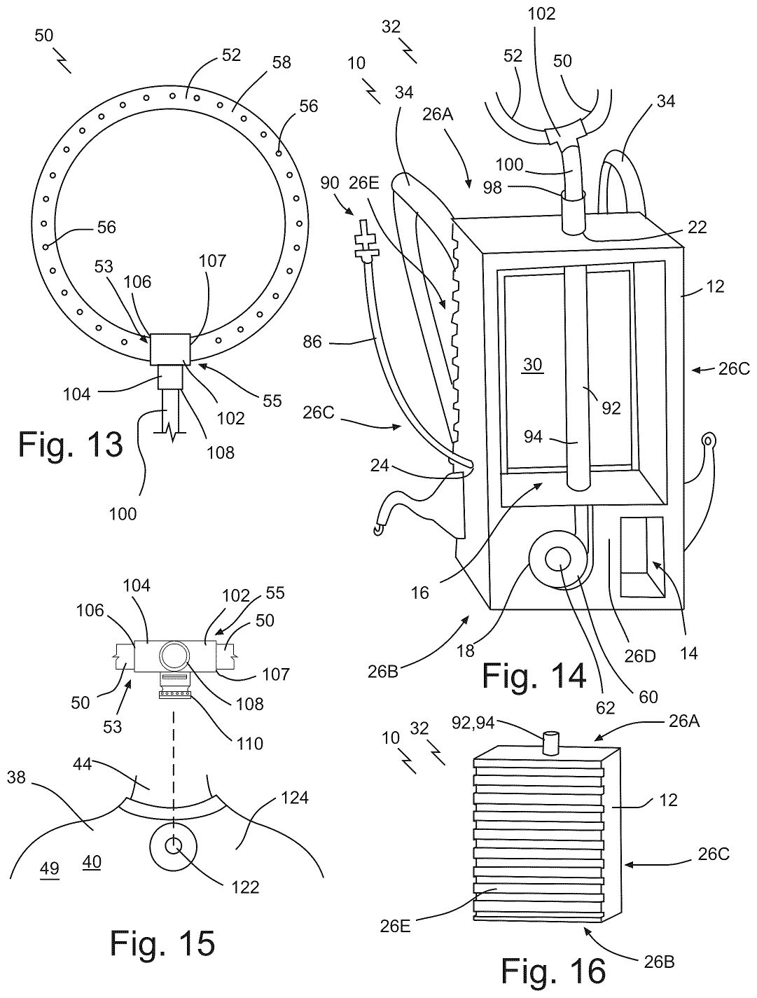

Referring to FIGS. 1-5 and 13, a neck collar tube 50 may be incorporated in system or apparatus 10 to deliver cooling air about a user's head 46. The neck collar tube 50 may define a ring shape, such as a circular shape, and may be worn around the neck 44 of a user 38. The series of outlet openings 56 may be spaced about a suitable periphery, such as the top periphery 58 of the neck collar tube 50, although in some cases openings 56 may be positioned elsewhere, such as in an inner periphery of the tube 50. The neck collar tube 50 may be hollow to provide an internal air passage to the openings 56. Referring to FIG. 3, the apparatus 10 may be structured to expel a vertical column of air 144, from the series of outlet openings 56, about a user's head 46 in use. A vertical column may refer to an effect where openings 56 direct air in a normal or near-normal (nominal deviations from vertical) direction out of the surface of tube 50 in an upward fashion, to provide a consistent curtain of air moving upward around all parts of the user's face and head. The vertical column of air 144 may cool the user 38 by providing cooled air directly to the user's head 46 and through evaporative cooling. The column of air 144 me produced to a degree, pressure, and flux sufficient to restrict or prevent insects such as mosquitos, horseflies, black flies, and other biting insects from accessing and biting the neck 44, head 46 and face 47 of the user 38.

Referring to FIGS. 1-5 and 13, the neck collar tube 50 may comprise a suitable material, such as latex. Latex tubing is a flexible and durable type of tubing widely used in various industries for its unique properties. Latex is made from natural latex rubber, which offers excellent elasticity, resilience, and resistance to abrasion. Latex's high stretchability allows for easy installation and secure attachment to connectors or fittings. Various properties of latex tubing make it inherently suitable for applications requiring flexibility and airtightness, such as in medical devices, laboratory equipment, and pneumatic systems. Latex tubing can be sterilized and is compatible with various chemicals and fluids, making it versatile for use in diverse environments. However, it is important to note that latex tubing may cause allergic reactions in individuals with latex allergies, and alternative materials should be considered in such cases.

Referring to FIGS. 1-3, 12 and 14, one or more wearable air mover or air pump, such as air mover 60, may be selected to provide personal cooling or enhance air circulation in specific applications of apparatus 10. Wearable air movers may be compact and lightweight, allowing them to be worn on the body or attached to clothing or equipment. Wearable air movers, such as air mover 60, may be equipped with small, high-performance fans or blowers that generate a focused stream of airflow. Air mover 60, may be controlled by a suitable controller 138, and may feature adjustable airflow settings and may be powered by rechargeable batteries for convenience and portability. Air mover 60 and apparatus 10 may be used in industries such as sports, outdoor activities, and personal protective equipment (PPE). Athletes, workers, tourists, and outdoor enthusiasts may wear apparatus 10 to cool themselves during intense physical activities, reducing the risk of heat-related stress. In industrial settings, workers may utilize the wearable air movers within their PPE to improve air circulation and maintain a comfortable working environment in confined or poorly ventilated spaces. apparatus 10, by incorporating air mover 60, may provide a hands-free solution, allowing individuals to benefit from targeted airflow while keeping their hands free for other tasks.

Referring to FIGS. 1-3, 12 and 14, the air mover 60 may be a suitable pump, such as a low-profile air mover. Low-profile air movers, also known as low-profile fans or low-clearance air movers, may be compact and lightweight devices designed to provide targeted airflow in confined spaces or areas with limited vertical clearance. Low-profile air movers may be specifically engineered with a low-profile design to fit in tight or shallow spaces, such as relatively flat compartments in a backpack as shown, where traditional fans or blowers may not be suitable. Despite their compact size, low-profile air movers may generate considerable airflow, effectively moving and circulating air within the designated area. Low-profile air movers may feature multiple speed settings and adjustable airflow directions to provide flexibility and control over the airflow. The compact design and lightweight nature of the low-profile air movers may facilitate easy transportation and positioning in various work environments.

Referring to FIGS. 1-3, 12 and 14, the air mover 60 may have a suitable structure. The air mover 60 may be housed in a slot 18, such as a pouch, pocket, or netting, within the housing 12. The air mover 60 may include an air mover housing 68. The housing 68 may define an inlet 62 and an outlet 64 of the air mover 60. The inlet and outlet may be in line as shown, or one may be an axial inlet/outlet and the other a radial or tangential inlet outlet, respectively. The inlet 62 of the air mover 60 may extend through an inlet 20 defined by the housing 12, which may allow the inlet 62 to draw air from an external environment, as shown by arrows 142. The housing 68 may comprise vanes 66. A motor may drive the vanes 66, such as an electrical motor. The vanes 66 may rotate around an axis 70 in order to draw air into the apparatus 10. The axis 70 may be perpendicular or normal to a user's back in as shown, or in another configuration. The outlet 64 of the air mover 60 may be connected to an inlet 96 of the air conduit 92, which may allow air to move through the air mover 60 and into the air conduit 92.

Referring to FIGS. 1-2, and 14, the air conduit 92 may comprise a suitable structure. The air conduit 92 may receive air from the air mover 60 through its inlet 96. The air conduit 92 may extend through the interior 30 of the housing 12, for example through or in close proximity to the drinking reservoir pouch 16. In some cases, the air conduit 92 is aligned to run parallel adjacent a spine of the user. The air conduit 92 may extend through an outlet 22 within the top surface 26A of the housing 12. The air conduit 92 may comprise a rigid standpipe 94. The conduit 92 may connect via a manifold 102 to the neck collar tube 50, or in another suitable fashion such as a direct connection. An outlet 98 of the rigid stand pipe 94 may connect to a flexible or resilient tube 100 or the air conduit 92. The malleability of the tube 100 may allow for the user 38 to maintain a standard range of motion and may reduce stress on the rigid stand pipe 94 and the neck collar tube 50 when the user is moving. The flexible tube 100 may connect the rigid stand pipe 94 to the neck collar tube 50.

A heat exchanger is a device that facilitates the efficient transfer of thermal energy between two fluids at different temperatures. A heat exchanger may have a series of tubes or channels through which the fluids flow, allowing for heat exchange without direct contact between the fluids. A heat exchanger may operate on the principle of maintaining a high surface area for maximum heat transfer. As the fluids flow in separate passages, heat may be transferred from the hot fluid to the cooler fluid through conduction. The heat exchanger process may be aided by factors such as the flow rate, surface area, and thermal conductivity of the materials used. A heat exchanger may be designed in various configurations, including shell and tube, plate, or finned tube arrangements, depending on the specific application and requirements.

Referring to FIGS. 1-2, and 14, the air conduit 92 may be configured for heat exchange with the fluids in the reservoir 72. In some cases, the conduit 92 may be configured for non-mixing heat exchange with the drinking fluid reservoir 72. The air conduit 92 may be at least partially inset within an indented conduit slot 84, such as a vertical slot, defined by an exterior wall 85 of the drinking fluid reservoir 72. The drinking fluid reservoir 72 may in use be filled with cold liquid or solids, such as ice, which may undergo heat exchange with the air within the air conduit 92 during use to cool the air passing to the tube 50. The indented conduit slot 84 of the reservoir 72 may be one example of a way to increase the surface area contacting the air conduit 92, which may allow for increased heat exchange between the fluid in the reservoir 72 and the air in the air conduit 92. The air conduit 92 may comprise a suitable material which allows for efficient heat exchange between the air in the air conduit 92 and the fluid in the reservoir 72, such as aluminum, or other thermally conductive materials.

Referring to FIGS. 2, 4, 6, 10-11, 13 and 16, the apparatus 10 may comprise an air manifold 102. An air manifold may include a component that is used in pneumatic systems, for example to distribute air or gases to multiple outlets or devices. The air manifold 102 may serve as a central hub which receives the air supply from the flexible tube 100 of the air conduit 92 and divides the air supply into multiple pathways. The manifold 102 may receive air through the air inlet 108. The manifold 102 may have one or more outlets, at least one of which feeds air to the neck collar tube 50. The manifold 102 may consist of a fitting body 104 with multiple outlets where air connections can be made, for example a first outlet 106, a second outlet 107 and a third outlet 110. Each outlet may form a branch of the manifold. Each outlet may be designed to connect to individual hoses, tubes, manifolds, or fittings, and that may lead to different pneumatic devices, actuators, or outlets to the body. The manifold 102 may ensure distribution of air pressure across all outlets, allowing consistent and synchronized operation of the connected pneumatic components. The first outlet 106 and the second outlet 107 may feed air to a first end 53 and a second end 55 of the neck collar tube 50, respectively.

Referring to FIGS. 210 and 11, the air manifold 102 may comprise a valve 112 or other flow control device such as a regulator to control the airflow. Valve 112 may have a suitable structure, such as that of a gate valve as shown, with a gate body 114, which may be used to close or open the third outlet 110 or one or more of the outlets. The body 114 of the valve 112 may be structured to travel within a corresponding slot in the manifold. The manifold may define a travel slot 118, and that may interact with a pin 116 of the gate body 114 to set valve travel limits between an open or closed position. In the example shown, the gate body 114, which may be a flat plate such as a stadium of rotation as shown, that closes off the third outlet 110 perpendicular to the other outlets.

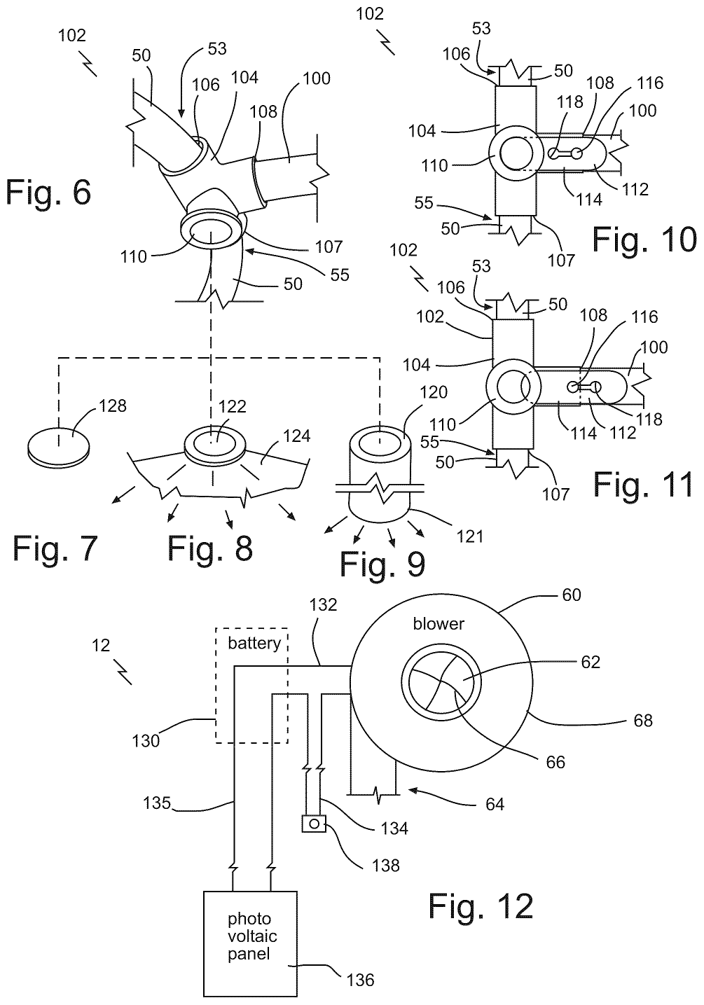

Referring to FIGS. 6-11 and 15, the manifold 102 may be structured to feed air to other parts of the body 49 of a user 38 beyond just the neck and head of the user. The manifold 102 may define at least one outlet of the one or more outlets connected to feed a body cooling tube 120, for example via the third outlet 110. A valve 112, if present, may be set to the open position, to allow air to flow be expelled from the third outlet 110. The outlet 110 or tube 120 may be connected to various fittings. The outlet 110 may connect to the body cooling tube 120 (FIG. 9), which may be manually positioned by the user 38 to feed air to the front 42 and back 40 of the user 38, or to other areas of the body as directed by the user. The tube 120 may have suitable clips or mounts to permit selective positioning of the tube 120 outlet 121. The outlet 110 may connect to an inlet 122 defined within an article of the user's clothing 124 (FIG. 8), for example a shirt, cape, sheet, poncho, or other article of clothing on the user 38 in order to provide air flow to the body 49 of the user 38. The user 38 may also be provided with a mechanism for closing the outlet 110, such as using a cap 128 (FIG. 7) when not in use, and/or the valve 112 may be moved to the closed position to restrict air through outlet 110.

Referring to FIGS. 1-3 and 13, the apparatus 10 may comprise a backpack-housed water bladder system, such as the drinking fluid reservoir 72. A fluid inlet (not shown) may be at a suitable location such as a top 80 of the reservoir. An outlet 82 for a drinking tube 86 may be at a suitable location such as a bottom 81 of the reservoir 72. The interior 74 of the drinking fluid reservoir 72 may be filled with a suitable drinking fluid, or a solid that will melt into a suitable fluid, such as ice 76 and water 78. In some cases, the reservoir 72 or housing may include another PCM such as provided in a sealed ice-pack, which may or may not be in the reservoir 72. The presence of ice 76 within the reservoir 72 may increase the heat exchange abilities of the fluid within the reservoir 72, as the latent heat of melting from transitioning the ice 76 to water via heat from the air in conduit 92 may increase the amount of heat that the fluid is able to absorb from the air in the air conduit 92 relative to liquid water alone. The inlet 88 of the drinking tube 86 may be connected to the outlet 82 of the reservoir 72. An outlet 90 of a drinking tube 86 may allow the user 38 to drink from the reservoir 72, and may comprise a valve, such as a bite valve, to prevent unwanted leaking from the reservoir 72. The drinking tube 86 may extend through an outlet 24 the housing 12. The outlet 24 may be positioned on the top surface 26A or side surface 26C of the housing 12. The drinking tube 86 may attach to a convenient part of the backpack 32, for one of the straps 34.

Referring to FIGS. 1-2, 12 and 14, the personal cooling apparatus 10 may comprise a suitable power source. A battery 130 may be provided, and may be able to be removed from the apparatus 10 in order to be replaced or recharged. In some cases, charging apparatus is included, such as having an electrical outlet or charging cord that can be connected to battery 130. The battery 130 may be connected to the air mover 60 through a suitable mechanism, such as via electrical leads 132. The apparatus may comprise a controller 138, which may be connected to the battery through electrical leads 134. The controller 138 may comprise one or more suitable controls, such as buttons 140, which may allow the user to turn the air mover 60 on and off and to set the speed of the air mover 60, or carry out other functions or adjustments of function parameters. The apparatus 10 may comprise other power sources, such as a photovoltaic panel, for example a solar panel 136. A solar panel 136 may be connected to the battery 130 through electrical leads 135. The solar panel 136 may be used to provided charge to the battery 130.

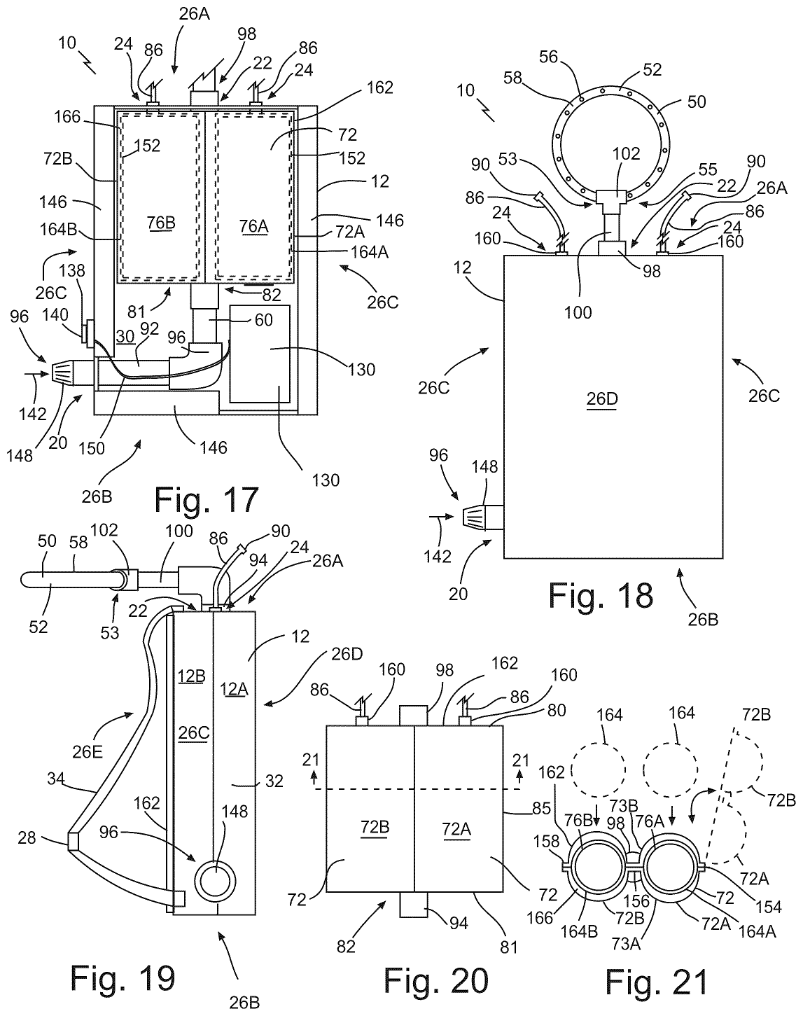

Referring to FIGS. 17-21, another embodiment of a personal cooling apparatus 10 is illustrated. In the example shown, the air conduit 92 forms an envelope 162 that encloses the drinking fluid reservoir 72. Inner surfaces of the envelope 162 and outer surfaces of the drinking fluid reservoir 72 may define a peripheral air passage plenum 166 around the drinking fluid reservoir 72. In the example shown, the plenum 166 is defined as a clearance around a periphery of the reservoir 72. The drinking fluid reservoir 72 may comprise one or more bottles, or other compartments, such as bottles 164A and 164B. The bottles 164A/164B may be cylindrical bottles and may be filled with water and frozen prior to use. The bottles 164A and 164B may include outlets 24. One or more filters 160 may be provided in the event that the water used is not potable water. Other outlets may be provided for connecting to drinking tubes 86. Corresponding apertures may be defined in the envelope 162 to pass the outlets 24. The envelope 162 may be shaped to form a conformal enclosure that forms a suitable but relatively narrow peripheral clearance around the one or more bottles. In the example shown, the envelope 162 forms a clamshell, for example with first and second jaw or clamshell parts 72A and 72B, that enclose the two or more cylindrical bottles 164A and 164B when in a closed position (solid lines FIG. 21). The clamshell design may be provided for case of construction, with both parts 72A and 72B being permanently secured and sealed together prior to use, to form a hermetically sealed module that may be added or removed from the apparatus to replenish the ice supply without separating the bottles and envelope. Each bottle 164A and 164B may include a cap, such as a closcable cap. The parts 72A and 72B may also be opened in some cases, for example to permit a user to add or remove the bottles 164A and 164B. A lock (not shown) may be provided to secure the envelope 162 in the closed position. By providing a conformal inner plenum 166 that effectively passes air in the conduit 92 along the exterior surfaces of the reservoir 72 and through the interstices between the reservoir 72 and conduit 92, surface area contact between the cold ice and the hot air is maximized, increasing heat transfer. The bottles 164A and 164B may be shaped for increased surface area contact, for example they may be ridged or finned. Each bottles 164A and 164B may form a respective ice block 76A and 76B, which as it melts, may form liquid water that can be withdrawn via one or more tubes 86. In the example shown, dual tubes 86 are provided, for various purposes, such as a second water source for the user or for a third party such as an individual the user is assisting. One or more air filters 148 may be provided via the air mover or air conduit, in this case on the inlet 62, to provide clean air to the user, which may be advantageous if the air is contaminated, for example during a forest fire. A suitable air mover 60 may be used, such as an inline pump as shown. A control line 150 may run between a battery 130 and controller 138 to allow the user to use buttons 140 or other controls, such as a speed control dial to operate the unit. A thermostat control may be provided in conjunction with a temperature sensor. Foam or other pads 162 may be provided on an exterior of the housing for comfort. The housing 12 may be provided in two or more parts 12A and 12B that may be separated or otherwise opened and locked together to permit access by the user to the interior to swap out and install the ice block system. The housing 12 may be insulated to minimize heat transfer between the ice and the ambient environment, for example using insulation 146 such as foam padding around the periphery of the interior of the housing 12. The example shown uses frozen water containers, loosely enveloped in plastic to maximize air contact with the outside surface of the water containers, creating a reduced temperature/cooled air flow. Each of the two tanks may have a drinking tube that draws melted water into the containers. In some cases, the apparatus 10 or the modular envelope ice block system may be used as a portable air conditioning unit, for example by operating the unit in a room to cool air without need for a hot air exhaust system.

In the claims, the word “comprising” is used in its inclusive sense and does not exclude other elements being present. The indefinite articles “a” and “an” before a claim feature do not exclude more than one of the feature being present. Each one of the individual features described here may be used in one or more embodiments and is not, by virtue only of being described here, to be construed as essential to all embodiments as defined by the claims.

1. A personal cooling apparatus comprising:
an air mover;
a drinking fluid reservoir;
an air conduit connected to an outlet of the air mover and configured for heat exchange with the drinking fluid reservoir; and
a neck collar tube connected to the air conduit and defining a series of outlet openings spaced about a periphery of the neck collar tube.

2. The personal cooling apparatus of claim 1 in which the neck collar tube defines a ring, in which the series of outlet openings are spaced about a top periphery of the neck collar tube.

3. The personal cooling apparatus of claim 1 in which the neck collar tube comprises latex.

4. The personal cooling apparatus of claim 1 in which one or more of:
the personal cooling apparatus is configured to expel a vertical column of air, from the series of outlet openings, about a user's head in use; and
the air conduit is configured for non-mixing heat exchange with the drinking fluid reservoir.

5. The personal cooling apparatus of claim 1 in which the air conduit forms an envelope that encloses the drinking fluid reservoir, with inner surfaces of the envelope and outer surfaces of the drinking fluid reservoir defining a peripheral air passage plenum around the drinking fluid reservoir.

6. The personal cooling apparatus of claim 1 in which:
the drinking fluid reservoir comprises one or more bottles; and
the envelope is shaped to form a conformal enclosure around the one or more bottles.

7. The personal cooling apparatus of claim 6 in which:
the one or more bottles comprise two or more cylindrical bottles; and
the envelope forms a clamshell that encloses the two or more cylindrical bottles when in a closed position.

8. The personal cooling apparatus of claim 1 in which the air conduit comprises a rigid standpipe that connects via a manifold to the neck collar tube.

9. The personal cooling apparatus of claim 1 in which the air conduit connects via a manifold to the neck collar tube, and the manifold has one or more outlets, at least one of which feeds the neck collar tube.

10. The personal cooling apparatus of claim 9 in which the manifold comprises a fitting with first and second outlets feeding first and second ends of the neck collar tube.

11. The personal cooling apparatus of claim 9 in which one or more of:
at least one outlet of the one or more outlets is connected to feed a body cooling tube; and
the manifold comprises a valve.

12. The personal cooling apparatus of claim 1 further comprising one or more drinking fluid tubes connected to the drinking fluid reservoir.

13. The personal cooling apparatus of claim 1 in which the air mover or air conduit comprise one or more air filters.

14. The personal cooling apparatus of claim 1 further comprising a housing mounting the air mover, drinking fluid reservoir, and air conduit.

15. The personal cooling apparatus of claim 14 in which the housing comprises a backpack with shoulder straps.

16. The personal cooling apparatus of claim 1 further comprising a controller for the air mover.

18. The personal cooling apparatus of claim 1 in which the drinking fluid reservoir contains ice.

19. A method comprising operating the air mover of the personal cooling apparatus of claim 1 to pump air through the personal cooling apparatus to cool a user.

20. A method comprising operating an air mover, of a personal cooling apparatus worn by a user, to:
move air from an external environment into heat exchange with a drinking fluid reservoir, of the personal cooling apparatus, to cool the air; and
expel the air upward around a face of the user via a series of outlet openings spaced about a periphery of a neck collar tube, of the personal cooling apparatus.
